(19) United States
(12) Patent Application Publication
(10) Pub. No.: US 2023/0322838 A1
(43) Pub. Date: Oct. 12, 2023

(54) DIOSMIN PREPARATION METHOD

(71) Applicant: LES LABORATOIRES SERVIER, SURESNES CEDEX (FR)

(72) Inventors: Bruno SCHIAVI, Gruchet Ie Valasse (FR); Philppe BESCOND, Saint-Vrain (FR); Patrick MOUCHET, Ectot-L'auber (FR)

(21) Appl. No.: 18/004,468

(22) PCT Filed: Jul. 8, 2021
(86) PCT No.: PCT/EP2021/068970

§ 371 Date: Jan. 6, 2023

(30) Foreign Application Priority Data
Jul. 9, 2020 (EP) 20315345.7

(51) Int. Cl. C07H 17/07; C07H 1/00

(52) U.S. Cl. CPC C07H 17/07; C07H 1/00

(57) ABSTRACT

Process for the preparation of diosmin.

The present invention relates to a process for the preparation of diosmin.

Diosmin is the compound of formula (I):

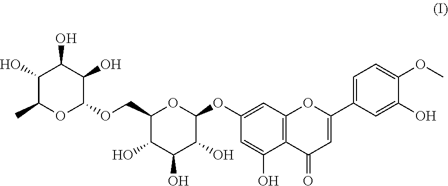 [SMILES: COc1ccc(-c2cc(=O)c3c(O)cc(O[C@@H]4O[C@H](CO[C@@H]5O[C@@H](C)[C@H](O)[C@@H](O)[C@H]5O)[C@@H](O)[C@H](O)[C@H]4O)cc3o2)cc1O]

Diosmin is used in the treatment of venous diseases, such as chronic venous insufficiency or haemorrhoidal diseases.

It is also the major component of micronised purified flavonoid fraction, or MPFF (Daflon®).

Diosmin is synthesised by oxidation of hesperidin. Hesperidin is the compound of formula (II):

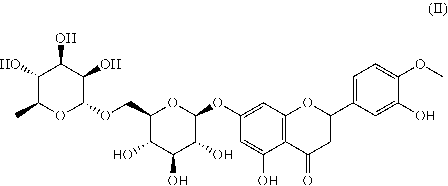 [SMILES: COc1ccc(C2CC(=O)c3c(O)cc(O[C@@H]4O[C@H](CO[C@@H]5O[C@@H](C)[C@H](O)[C@@H](O)[C@H]5O)[C@@H](O)[C@H](O)[C@H]4O)cc3O2)cc1O]

Hesperidin is obtained from natural substances (oranges). The diversity of oranges used leads to hesperidins of unequal purity, containing other flavonoids with variable contents. In particular, the hesperidin may contain up to 4% of isonaringin, which is converted to isorhoifolin by oxidation.

Therefore, diosmin generally contains other flavonoids, some of which originate from the oxidation of the flavonoids present in the initial hesperidin, and others are reaction by-products.

Given the pharmaceutical interest of diosmin, it is essential to obtain it with an excellent yield and the required purity, irrespective of the source of hesperidin.

The specifications imposed by the European Pharmacopoeia are the following:

| | Diosmin specifications |
|---|---|
| Substances | (European Pharmacopoeia) |
| Diosmin | 90.0 to 102.0% |
| Hesperidin | <4.0% |
| Diosmetin | <2.0% |
| Isorhoifolin | <3.0% |
| Linarin | <3.0% |
| 6-Iododiosmin | <0.6% |

In particular, it is essential that the diosmin obtained contain less than 0.6% of 6-iododiosmin and less than 3.0% of isorhoifolin.

Processes for the preparation of diosmin from hesperidin have been described in the literature. FR2311028 describes a process for obtaining diosmin by acetylation of hesperidin followed by oxidation of the acetylated hesperidin by bromination, basic hydrolysis and isolation. The crude diosmin thus obtained is purified by a retreatment step using pyridine.

This method is not ideal, since the yield is only 65%. Furthermore, it uses pyridine, a class 3 carcinogenic solvent.

Patent application WO2016/124585 has the advantage of not using organic solvents such as pyridine. However, the process that is described therein does not make it possible to obtain diosmin with the required purity when the hesperidin used contains a large amount of isonaringin.

One of the problems for the present invention was to minimise the content of 6-iododiosmin in the diosmin obtained, while dispensing with the use of class 3 solvents such as pyridine. Another problem for the present invention was to minimise the content of isorhoifolin in the diosmin obtained, while dispensing with the use of class 3 solvents such as pyridine, while starting from a hesperidin containing up to 4% of isonaringin.

More specifically, the present invention relates to a process for the preparation of diosmin by
a) acetylation of hesperidin,
b) oxidation of the acetylated hesperidin to acetylated diosmin by an iodine donor, at a temperature of from 90 to 120° C.,
c) heating of the acetylated diosmin, in an autoclave at a pressure of from 5 to 8 bar, under reflux of an alcohol such as methanol, ethanol or isopropanol, in the presence of a base chosen from sodium acetate or potassium acetate, sodium hydroxide, potassium hydroxide or lithium hydroxide, potassium carbonate, sodium methanolate or sodium ethanolate, alone or as a mixture with another of these bases, then
d) deprotection of the acetylated diosmin to diosmin by heating in the presence of a base chosen from sodium hydroxide, potassium hydroxide or lithium hydroxide, potassium carbonate, sodium methanolate or sodium ethanolate, alone or as a mixture with sodium acetate or potassium acetate,
e) purification by base/acid treatment.

According to one embodiment of the present invention, the diosmin obtained contains other flavonoids such as hesperidin, isorhoifolin, linarin or diosmetin.

According to one embodiment of the present invention, the acetylation step (a) is carried out by reacting hesperidin with acetic anhydride and potassium or sodium acetate.

The acetylation reaction is preferably carried out at a temperature between 40° C. and 135° C.

The amount of acetic anhydride is preferably between 8 and 10 molar equivalents relative to the hesperidin used.

The iodine donor used in the oxidation step (b) is preferably chosen from NaI/H₂O₂, KI/H₂O₂, TBAI/H₂O₂ and NaI/I₂ (pref. 9/1)/H₂O₂.

The amount of NaI is preferably from 0.05 to 0.20 molar equivalent relative to the hesperidin used.

The amount of hydrogen peroxide is preferably from 1.0 to 1.2 molar equivalents relative to the hesperidin used.

According to one embodiment of the present invention, the acetylated diosmin obtained at the end of the oxidation step (b) is isolated, preferably by precipitation in water before being used in step c).

According to one embodiment of the present invention, the base used for step c) is an aqueous solution of sodium hydroxide or potassium hydroxide, an aqueous solution of sodium acetate or potassium acetate, or a mixture of sodium hydroxide or potassium hydroxide and sodium acetate or potassium acetate in aqueous solution.

The sodium acetate or potassium acetate used for step c) can be generated in situ by neutralisation of the residual acetic acid present in the acetylated diosmin with sodium hydroxide or potassium hydroxide.

The amount of base used in step (c) is preferably between 0.5 and 2.5 molar equivalents relative to the hesperidin used.

According to one embodiment of the present invention, the base added to the deacetylation step (d) is sodium hydroxide or potassium hydroxide.

The amount of base added to the deacetylation step (d) is preferably between 2 and 4.5 molar equivalents relative to the hesperidin used.

According to one embodiment of the present invention, the base/acid treatment (step e) is carried out by passing into solution in water in the presence of a base such as sodium hydroxide, then precipitation by salification with an acid, such as sulfuric acid.

The following examples illustrate the invention.

### ABBREVIATIONS

mol eq molar equivalents (relative to the hesperidin)
HPLC high performance liquid chromatography
m/m ratio expressed as mass/mass
TBAI tetra-n-butylammonium iodide
vol volume equivalents (relative to the hesperidin)

### EXAMPLE 1: DIOSMIN

Step A: Acetylated diosmin

Introduce into a reactor between 20-25° C. potassium acetate (98.6 mmol) and acetic anhydride (2996.2 mmol).

Heat the suspension, while stirring, to 40° C. then introduce hesperidin (2×163.8 mmol; HPLC titer: 91.3%, isonaringin 3.8%). Continue stirring while heating at 40° C., then heat to 132° C. in 45 min while stirring. The mixture changes to a clear solution at the end of the heating. Stir the solution for 60 min at 132° C. then cool to 105° C.

Introduce aqueous sodium iodide solution (33 mmol in 20 g of water). At 105° C., pour in hydrogen peroxide 35% (341.5 mmol) stabilised with 0.1% sulfuric acid.

Stir for 30 min at 105° C. then cool to 100° C. while stirring and precipitate in a beaker containing water (approx. 7 vol), with mechanical stirring at 20-40° C.

After 30 min of stirring between 20-40° C., filter in vacuo, wash the cake with water (9 vol; then 2×2 vol). Expurge for 16 h in vacuo between 20-25° C.

Step B: Diosmin

Introduce into an autoclave the acetylated diosmin obtained in Step A and methanol (3.5 vol). Place under stirring then heat under reflux at a pressure of 5 bar. After 15 min under reflux, introduce sodium hydroxide as a 30% aqueous solution (1.2 mol eq). Heat under reflux for 30 min then cool to 50° C. at normal pressure and introduce sodium hydroxide as a 30% aqueous solution (2.4 mol eq). After 2 h at 50° C., cool to 20° C. then filter and wash the cake with methanol (2×3 vol).

Dissolve the crude diosmin in 2.5 mol eq of sodium hydroxide and water (2.5 vol) at 20° C.

Add sulfuric acid to adjust the pH to between 2 and 4. Maintain for 30 min at 20° C., filter, wash twice with water (2×5 vol) and dry.
Yield starting from hesperidin: 83.8%
Purity (HPLC): 90.6%
Content of 6-iododiosmin: 0.3%
Content of isorhoifolin: 2.0%.

### EXAMPLE 2: DIOSMIN

Step A: Acetylated diosmin

Introduce into a reactor between 20-25° C. potassium acetate (207.1 mmol) and acetic anhydride (6291.9 mmol).

Heat the suspension, while stirring, to 100° C. then introduce hesperidin (5×137.6 mmol; HPLC titer: 91.7% and isonaringin 3.6%). Continue stirring while heating at 100° C., then heat to 132° C. in 15 min while stirring. The mixture changes to a clear solution at the end of the heating. Stir the solution for 120 min at 132° C. then cool to 105° C.

Introduce aqueous sodium iodide solution (68.8 mmol in 40 g of water). At 105° C., pour in hydrogen peroxide 35% (717.1 mmol) stabilised with 0.1% sulfuric acid.

Stir for 30 min at 105° C. then cool to 100° C. while stirring and precipitate in a beaker containing water (approx. 7 vol), with mechanical stirring at 20-40° C.

After 30 min of stirring between 20-40° C., filter in vacuo, wash the cake with water (9 vol; then 2×2 vol). Expurge for 16 h in vacuo between 20-25° C.

Step B: Diosmin

Introduce into an autoclave the acetylated diosmin obtained in Step A and methanol (3.5 vol). Place under stirring then heat under reflux at a pressure of 5 bar. After 15 min under reflux, introduce sodium hydroxide as a 30% aqueous solution (1.55 mol eq). Heat under reflux for 30 min then cool to 50° C. at normal pressure and introduce sodium hydroxide as a 30% aqueous solution (2.4 mol eq). After 2 h at 50° C., cool to 20° C. then filter and wash the cake with methanol (2×3 vol).

Dissolve the crude diosmin in 2.5 mol eq of sodium hydroxide and water (2.5 vol) at 20° C. Add sulfuric acid to adjust the pH to between 2 and 4. Maintain for 30 min at 20° C., filter, wash twice with water (2×5 vol) and dry.
Yield starting from hesperidin: 81.2%
Purity (HPLC): 90.4%
Content of 6-iododiosmin: 0.29%
Content of isorhoifolin: 2.2%.

### EXAMPLE 3: DIOSMIN

Introduce into an autoclave the acetylated diosmin obtained in Step A of Example 1 and methanol (3.5 vol), add 2 mol eq of an aqueous solution of potassium acetate then heat under reflux at a pressure of from 7 to 8 bar. Next, cool to 50° C. and introduce an aqueous solution of potassium hydroxide (4.2 mol eq). After contact at 50° C., cool to 20° C., then filter and wash with methanol (2×1.5 vol).

Dissolve the crude diosmin in 2.5 mol eq of sodium hydroxide and water (2.5 vol) at 20° C. Add sulfuric acid to adjust the pH to between 2 and 4. Maintain for 30 min at 20° C., then filter, wash twice with water (2×5 vol) and dry.
Yield starting from hesperidin: 87.7%
Purity (HPLC): 90.1%
Content of 6-iododiosmin: not detected (<0.10%)

### EXAMPLE 4: ACETYLATED DIOSMIN WITH DIFFERENT IODINE DONORS

Into a 25-ml three-necked flask equipped with an ovoid stirrer and a syringe driver, introduce 10 g of hesperidin, 0.5 g of potassium acetate and 14 ml/15.6 g of acetic anhydride. Gradually bring the temperature to 132° C. and leave at 130° C. for 1 h.

Cool to approximately 90° C. then introduce 0.322 g of sodium iodide or the equivalent iodine donor and 2.258 g of water. Heat to 105° C. then add hydrogen peroxide 35% (1.1835 ml/1.645 g) and 5.161 g of water.

| Test | 3a | | 3b | | 3c | | 3d | |
|---|---|---|---|---|---|---|---|---|
| Hesperidin | 10 | g | 2 | g | 2 | g | 2 | g |
| Acetic | 9.146 | mol eq | 9.146 | mol eq | 9.146 | mol eq | 9.146 | mol eq |
| anhydride | | | | | | | | |
| Potassium | 0.327 | mol eq | 0.327 | mol eq | 0.327 | mol eq | 0.327 | mol eq |
| acetate | | | | | | | | |
| 35% H₂O₂ | 1.036 | mol eq | 1.033 | mol eq | 1.033 | mol eq | 1.033 | mol eq |

| | | | | | | | |
|---|---|---|---|---|---|---|---|
| H₂SO₄ | / | 0.00109 | mol eq | 0.00109 | mol eq | 0.00109 | mol eq |

| | | | | |
|---|---|---|---|---|
| Iodine donor | NaI | KI | TBAI | NaI/I₂ 9/1 |
| | 0.133 mol eq | 0.133 mol eq | 0.133 mol eq | 0.120/0.013 mol eq |
| Acetylated | 94% | >97% | >98% | >97% |
| diosmin | | | | |

### EXAMPLE 5: DIOSMIN WITH DIFFERENT BASES

Introduce into an autoclave the acetylated diosmin obtained in Step A of Example 2 and methanol (3.5 vol). Place under stirring then heat under reflux at a pressure of 7 bar. After 15 min under reflux, introduce the base as a 30% aqueous solution (1.2 mol eq). Heat under reflux for 30 min then cool to 50° C. at normal pressure and introduce the base as a 30% aqueous solution (2.4 mol eq). After 2 h at 50° C., cool to 20° C. then filter and wash the cake with methanol (2×3 vol).

| Test | 4a | 4b | 4c | 4d | 4e |
|---|---|---|---|---|---|
| Base | CH₃ONa | NaOH | LiOH | KOH | K₂CO₃ |
| Yield/Hesperidin used | 84% | 84% | 85% | 84% | 80% |
| Diosmin | 89.8% | 90.9% | 90.4% | 90.8% | 92.4% |
| Isorhoifolin | 2.6% | 2.0% | 2.1% | 2.0% | 2.1% |
| 6-Iododiosmin | 0.45% | 0.46% | 0.42% | 0.62% | 0.54% |

### EXAMPLE 6 (COMPARATIVE): REPRODUCTION OF THE PROCESS OF WO 2016/124585

40 g of acetic anhydride, 0.75 g of potassium acetate and 30 g of hesperidin (purity 91.3%; isonaringin 3.8%) are introduced into a reactor. The reaction medium is then heated to 115-120° C., maintaining this temperature for one hour approximately, and then the medium is cooled to 60-70° C.

A solution of sodium iodide (0.9 g) in water (6 ml) is added, and the reaction medium is heated to reflux. Then, a solution of 35 ml of 5.4% (by mass) hydrogen peroxide stabilised with sulfuric acid is added to the reaction medium, while maintaining the reflux. Next, the reaction medium is cooled to 40-50° C. and potassium hydroxide (10 g) is added to the reaction mixture; the pH is then 4. The mixture is then heated at 115-120° C. for 3 hours, and then cooled to 30° C.

The reaction mixture is added to a reactor containing a 2N aqueous sodium hydroxide solution (300 ml). After 1 h and 30 min, sulfuric acid is added until the pH reaches 7.5. The precipitate is then filtered and washed with water, to obtain wet crude diosmin.

The crude diosmin thus obtained is crystallised by dissolving it in an aqueous solution of sodium hydroxide, then by acidifying it with sulfuric acid until the product precipitates.

The solid is filtered, washed with water and dried.

Analysis (HPLC):

| | Percentage in | Diosmin |
|---|---|---|
| | the product | specifications |
| | of | (European |
| Substances | Example 5 | Pharmacopoeia) |
| Diosmin | 87.1% | 90.0 to 102.0% |
| Isorhoifolin | 3.6% | <3.0% |
| 6-Iododiosmin | 0.99% | <0.6% |

1-13. (canceled)

14. A process for the preparation of diosmin comprising the following steps:
a) acetylation of hesperidin,
b) oxidation of the acetylated hesperidin to acetylated diosmin by an iodine donor, at a temperature of from 90 to 120° C.,
c) heating the acetylated diosmin, in an autoclave at a pressure of 5 to 8 bar, under reflux of an alcohol, in the presence of a base selected from sodium acetate, potassium acetate, sodium hydroxide, potassium hydroxide, lithium hydroxide, potassium carbonate, sodium methanolate, andsodium ethanolate, and mixtures thereof, then
d) deprotection of the acetylated diosmin to diosmin by heating in the presence of a base selected from sodium hydroxide, potassium hydroxide, lithium hydroxide, potassium carbonate, sodium methanolate, and sodium ethanolate, alone or as a mixture with sodium acetate or potassium acetate,
e) purification by base/acid treatment.

15. The process according to claim 14, wherein the diosmin obtained contains other flavonoids.

16. The process according to either claim 15, wherein the diosmin obtained contains less than 0.6% of 6-iododiosmin and less than 3.0% of isorhoifolin.

17. The process according to claim 14, wherein the amount of acetic anhydride is between 8 and 10 molar equivalents relative to the hesperidin used.

18. The process according to claim 14, wherein the acetylation reaction (a) is carried out at a temperature between 40° C. and 135° C.

19. The process according to claim 14, wherein the iodine donor is selected from NaI/H₂O₂, KI/H₂O₂, TBAI/H₂O₂ and NaI/I₂/H₂O₂.

20. The process according to claim 19, wherein the iodine donor is NaI in an amount of from 0.05 to 0.2 molar equivalent relative to the hesperidin used.

21. The process according to claim 14, wherein the amount of hydrogen peroxide is from 1.0 to 1.2 molar equivalents relative to the hesperidin used.

22. The process according to claim 14, wherein the acetylated diosmin obtained at the end of the oxidation step (b) is isolated by precipitation in water before being used in step c).

23. The process according to claim 14, wherein the base used for step c) is sodium hydroxide or potassium hydroxide in aqueous solution, sodium acetate or potassium acetate in aqueous solution, or a mixture of sodium hydroxide or potassium hydroxide and sodium acetate or potassium acetate in aqueous solution.

24. The process according to claim 14, wherein the alcohol used for step c) is methanol, ethanol or isopropanol.

25. The process according to claim 14, wherein the amount of base used in step (c) is between 0.5 and 2.5 molar equivalents relative to the hesperidin used.

26. The process according to claim 14, wherein the amount of base added to the deacetylation step (d) is between 2 and 4.5 molar equivalents relative to the hesperidin used.
